(19) United States
(12) Patent Application Publication
(10) Pub. No.: US 2016/0375190 A1
(43) Pub. Date: Dec. 29, 2016

(54) PERITONEAL DIALYSIS SYSTEMS AND METHODS

(71) Applicant: Cook Medical Technologies LLC, Bloomington, IN (US)

(72) Inventors: Duane Blatter, Salt Lake City, UT (US); Kalub Hahne, West Lafayette, IN (US); Andrew Isch, West Lafayette, IN (US); Keith Milner, West Lafayette, IN (US)

(21) Appl. No.: 15/167,334

(22) Filed: May 27, 2016

Related U.S. Application Data
(60) Provisional application No. 62/167,809, filed on May 28, 2015.

(51) Int. Cl. A61M 1/28; A61M 1/16

(52) U.S. Cl. CPC A61M 1/287; A61M 1/1696; A61M 1/1672; A61M 2205/8206

(57) ABSTRACT

Described are peritoneal dialysis systems and methods that involve the use of first and second stage filtration of a used dialysate withdrawn from the peritoneal space of a patient. The first filtration stage forms a first retentate containing an osmotic agent and a first permeate containing water and nitrogen-containing waste products of the patient. The second filtration stage acts on the first permeate to form a second retentate containing nitrogen-containing waste products of the patient and a second permeate containing water. At least some of the water from the second permeate is combined with the first retentate to form a regenerated peritoneal dialysis medium containing an amount of the osmotic agent. The regenerated peritoneal dialysis medium can be returned to the peritoneal space of the patient.

### REFERENCE TO RELATED APPLICATION

application claims the benefit of priority of U.S. Provisional Patent Application Ser. No. 62/167,809 filed May 28, 2015, which is hereby incorporated herein by reference in its entirety.

### BACKGROUND

For patients with chronic kidney disease who require renal replacement therapy, Peritoneal Dialysis (PD) has been shown to have significant advantages over hemodialysis. These advantages include lower overall costs, fewer hospitalizations and lower patient mortality. In addition, the process of peritoneal dialysis has been made relatively simple and most patients can learn the necessary skills. PD gives the patient greater flexibility in planning when to do dialysis.

Most patients receiving PD are treated with Automated Peritoneal Dialysis (APD). APD is a protocol of daily (usually nightly) treatment utilizing an automated pump. Typically multiple fill-drain cycles are programmed into the machine and occur automatically while the patient sleeps. Typically 12 to 15 liters are pumped into and out of the peritoneal space in 2 to 3 liter cycles with a specified dwell time between infusion and removal. The effluent is discarded into a drain.

Another implementation of PD is referred to Continuous Ambulatory Peritoneal Dialysis (CAPD). Patients receiving renal replacement therapy with CAPD manually infuse a defined amount of dialysate fluid into the peritoneal space at several times during the day, leaving the fluid for the dwell time and then manually draining into the drain bag.

In spite of its advantages, PD remains underutilized, particularly in the U.S. Only approximately 10% of kidney failure patients in the U.S. use PD for renal replacement. The limitations inherent to current implementations of PD contribute significantly to the underutilization. These limitations include:
The externalized catheter is inconvenient, causing limitations on showering, bathing and other activities of daily living.
There is a significant continuous risk of catheter tract infections and peritonitis and its complications.
Rapid transport of glucose across the peritoneal membrane in some patients renders PD ineffectual
The use of glucose based PD fluids that complicate blood sugar control in diabetic patients and cause weight gain in nearly all PD patients.
The complexity of the PD system, though moderate, can be intimidating for some patients and helpers.
While doing APD the patient is tethered to a bulky machine which limits mobility.
Large volumes of PD fluid must be delivered to and stored by the patient.

Various embodiments disclosed herein can eliminate or ameliorate one or more of the foregoing disadvantages with prior art systems. Various embodiments make PD easier to use and applicable to a larger percentage of chronic renal failure patients.

### SUMMARY

In certain aspects, provided are unique systems and methods for conducting peritoneal dialysis or regenerating a used dialysate solution. The methods and systems include filtering a used dialysate recovered from a peritoneal space of a patient to form a first retentate containing amounts of an osmotic agent, preferably a high molecular weight osmotic agent, of the dialysate solution and a permeate containing urea, creatinine and potentially other waste products from the patient, processing the permeate to recover at least some water therefrom, and then combining some or all of the recovered water with the first retentate containing the osmotic agent. Accordingly, in some embodiments herein, provide are peritoneal dialysis methods that include: (i) removing a peritoneal dialysis ultrafiltrate from a peritoneal space of a patient, the peritoneal dialysis ultrafiltrate containing an osmotic agent, water, and nitrogen containing waste products of metabolism of the patient; (ii) filtering particles from the peritoneal dialysis ultrafiltrate to form a pre-filtered peritoneal dialysis ultrafiltrate; (iii) passing the pre-filtered peritoneal dialysis ultrafiltrate through a first filter to form a first retentate containing an amount of the osmotic agent and a first permeate containing water and nitrogen containing waste products of the patient; (iv) passing the first permeate through a second filter to form a second retentate containing nitrogen containing waste products of the patient and a second permeate containing water; (vi) combining the second permeate with the first retentate to form a regenerated peritoneal dialysis medium containing an amount of the osmotic agent; and (vii) returning the regenerated peritoneal dialysis medium to the peritoneal space of the patient.

In other embodiments, provided are peritoneal dialysis apparatuses that include a catheter for removing a peritoneal dialysis ultrafiltrate from a peritoneum of a patient containing an osmotic agent (preferably a high molecular weight osmotic agent), water, and nitrogen containing waste products of metabolism of the patient; a filter arranged to filter particles from the peritoneal dialysis ultrafiltrate to form a pre-filtered peritoneal dialysis ultrafiltrate; a first filter arranged to filter the pre-filtered peritoneal dialysis ultrafiltrate to form a first retentate containing an amount of the osmotic agent and a first permeate containing water and nitrogen containing waste products of the patient; a second filter arranged to filter the first permeate to form a second retentate containing nitrogen containing waste products of the patient and a second permeate containing water; and a catheter for returning a regenerated peritoneal dialysis medium containing at least some of the water contained in the second permeate and the first retentate to the peritoneal space of the patient.

In still further embodiments herein, provided are methods for forming a regenerated peritoneal dialysis fluid. The methods include (i) filtering particles from a peritoneal dialysis ultrafiltrate of a patient, the peritoneal dialysis ultrafiltrate containing an osmotic agent (preferably a high molecular weight osmotic agent), water, and nitrogen containing waste products of metabolism of the patient, so as to form a pre-filtered peritoneal dialysis ultrafiltrate; (ii) passing the pre-filtered peritoneal dialysis ultrafiltrate through a first filter to form a first retentate containing an amount of the osmotic agent and a first permeate containing water and nitrogen containing waste products of the patient; (iii) passing the first permeate through a second filter to form a second retentate containing nitrogen containing waste products of the patient and a second permeate containing water; and (iv) combining at least some of the water contained in the second permeate with the first retentate to form a regenerated peritoneal dialysis medium containing an amount of the osmotic agent.

In still further embodiments herein, provided are methods for recapturing and reconstituting a high molecular weight peritoneal dialysis fluid. The methods include the steps of: filtering a dialysate fluid that has been removed from a peritoneal space of a patient to remove particulate material from the dialysate fluid, the dialysate fluid containing a high molecular weight component, and after said filtering, pumping the dialysate fluid into a high pressure segment of a first filtration chamber so that the dialysate fluid comes into contact with a first membrane having a molecular weight cutoff. The methods also include generating sufficient pressure in the high pressure segment of the first filtration chamber (e.g. with a pump) to result in transit of some of the water and solute molecules of the dialysate fluid that are below the molecular weight cutoff across the first membrane while the high molecular weight component of the dialysate fluid is constrained by the first membrane to the high pressure segment of the first filtration chamber, and wherein the water and solute molecules that transit across the first membrane exit the filtration chamber through a low pressure efferent lumen, and wherein the high molecular component constrained to the high pressure segment of the first membrane exits the filtration chamber with a fluid through a high pressure efferent lumen. The methods further include pumping the water and solute molecules that exit the filtration chamber through the low pressure efferent lumen into a high pressure segment of a second filtration chamber and separating water from nitrogen containing waste products of metabolism by a nanofiltration membrane, with the water crossing the nanofiltration membrane to a low pressure segment of the second filtration chamber and exiting the second filtration chamber through a low pressure efferent lumen, and the waste products that remained in the high pressure segment of the second filtration chamber exiting the second filtration chamber through a high pressure efferent lumen. Further included is a step of combining the water that exited the second filtration chamber through a low pressure efferent lumen with the fluid that exited the first filtration chamber through a high pressure efferent lumen to form a reconstituted peritoneal dialysis fluid. In some modes, the methods also include transporting the dialysate from the peritoneal space of the patient through a lumen of a peritoneal catheter and/or returning the reconstituted peritoneal dialysis fluid to the peritoneal space of the patient.

Additional embodiments of peritoneal dialysis methods and systems, as well as features and advantages attendant thereto, will be apparent from the descriptions herein.

### BRIEF DESCRIPTION OF THE DRAWINGS

FIG. 1 is a schematic representation of a wearable device for reconstitution of peritoneal dialysis fluid and its connections to the peritoneal space of a patient.

FIG. 2 is a schematic representation of an implantable device for reconstitution of peritoneal dialysis fluid and its connections to the peritoneal space and drainage into the ureter of a patient.

### DETAILED DESCRIPTION

For the purpose of promoting an understanding of the principles of the invention, reference will now be made to embodiments, some of which are illustrated with reference to the drawings, and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. Additionally, in the detailed description below, numerous alternatives are given for various components or features related to the described peritoneal dialysis systems, or to modes of carrying out steps or operations of methods for peritoneal dialysis or processing peritoneal dialysis fluids. It will be understood that each such disclosed alternative, or combinations of such disclosed alternatives, can be combined with the more generalized features discussed in the Summary above, or set forth in the Listing of Certain Embodiments below, to provide additional disclosed embodiments herein.

In various embodiments, peritoneal dialysis (PD) systems disclosed herein provide recapture and reconstitution of a high molecular weight (HMW) PD fluid. That fluid is then returned to the peritoneal space where it can act to draw additional waste metabolites and free water into the peritoneum.

Certain embodiments of PD systems described herein are small enough to be worn or implanted, and may allow continuous operation 24 hours per day. In certain embodiments, continuous operation is facilitated by a compact battery that is also small enough to be worn. In other embodiments, a semi-continuous operation can be implemented. In such operations, PD fluid can be allowed a dwell time in the peritoneal space of the patient, during which no PD fluid is withdrawn from the peritoneal space by the PD system (e.g. with the pump or pumps of the PD system de-energized or off during the dwell time). After the dwell time, the PD system is operated (e.g. by energizing or turning on a pump or the pumps of the PD system) to withdraw amounts of the used or spent PD fluid from the patient's peritoneal space, process the PD fluid to form a regenerated fluid as disclosed herein, and return the regenerated fluid to the peritoneal space of the patient. The withdrawal and return of these fluids from the peritoneal space can be simultaneous, e.g. operated in a continuous fluid loop from and to the peritoneal space. In embodiments operated in a cyclic or semi-continuous manner, the dwell time can range from about 1 hour to about 12 hours, from about 2 hours to about 6 hours, or from about 3 hours to about 4 hours. In addition or alternatively, the time over which the PD system is operated to withdraw and return fluids to the patient can range from about 1 hour to about 12 hours, from about 2 hours to about 6 hours, or from about 3 hours to about 4 hours. Also, whether operated in continuous, semi-continuous or other modes, it certain embodiments, the PD system and methods generate a liquid volume exchange in the peritoneal space of at least about 8 liters per day, or at least 10 liters per day, and typically in the range of about 8 to 20 liters per day or about 10 to 15 liters per day.

Certain embodiments operate with PD catheters that are, or are similar to, catheters that are already in common use. Most commonly used PD catheters comprise a soft silicone material with a single lumen and multiple side holes located at a curved or straight distal segment. Certain embodiments of PD systems disclosed herein operate with a dual lumen PD catheter, with one lumen for uptake from the peritoneal space and a second lumen for returning reconstituted fluid to the peritoneal space. Such catheters, while not in common clinical practice have been previously well described.

Embodiments of the PD systems disclosed herein can utilize high molecular weight (HMW) PD fluids. An example is Icodextrin, a high molecular weight starch dissolved in water. In particular, Icodextrin is a starch-derived, branched, water-soluble glucose polymer linked by α-(1→4) and less than 10% α-(1→6) glycosidic bonds. Its weight-average molecular weight is between 13,000 and 19,000 Daltons. Icodextrin is manufactured by Baxter Healthcare Corporation (sold under the tradename Extraneal) and is commonly used in current clinical practice. Icodextrin acts as a colloidal osmotic agent, although other high molecular weight osmotic agents can act as soluble, non-colloidal osmotic agents, and can also be used. Illustrative high molecular weight osmotic agents include glucose polymers (e.g Icodextrin), polypeptides (including for example albumin), dextran, gelatin and polycations. These or other high molecular weight osmotic components or agents typically have a weight average molecular weight of at least 10,000 Daltons, for example usually in the range of about 10,000 to about 350,000 Daltons and often in the range of about 10,000 to about 30,000 Daltons.

The PD fluid will typically include water, the osmotic agent(s), electrolytes such as sodium, calcium, potassium and/or magnesium, and a buffer. The buffer can for example be a lactate buffer, acetate buffer, or bicarbonate buffer. Other ingredients may also be present. The PD fluid will typically have a physiologically acceptable pH, for example in the range of about 5 to about 8. The PD fluid will also typically have an osmolality in the range of about 270 to 450 milliosmoles (mOsm), and more typically about 280 to about 350 mOsm. The osmotic agent can be present at any suitable concentration, and in some embodiments is present in the dialysis fluid or solution at a concentration of about 3 to about 20% by weight, or about 5 to about 15% by weight.

When a hyper osmolar PD fluid such as Icodextrin is introduced into the peritoneal space, water is drawn from the blood into the fluid until equilibrium is achieved. At the same time, nitrogen containing waste products of metabolism diffuse into the PD fluid. This mixture is referred to as an ultrafiltrate and contains urea, creatinine and a group of incompletely identified molecules of intermediate size.

Certain embodiments of the presently disclosed PD systems can employ a two stage filtering system (e.g. a two stage reverse osmosis filtering system) to recover and recycle the HMW PD fluid and return it to the peritoneal space. At the same time, the process yields a concentrated ultrafiltrate, separated from the HMW component containing the urea waste products that can be discarded. The first filtration stage separates the HWM starch or other osmotic agent from the remainder of the ultrafiltrate. The second stage filtration also employs reverse osmosis or other filtration to separate free water from the remainder of the ultrafiltrate. This free water is returned to the peritoneal space along with the HWM component of the first stage and the concentrated ultrafiltrate is discarded.

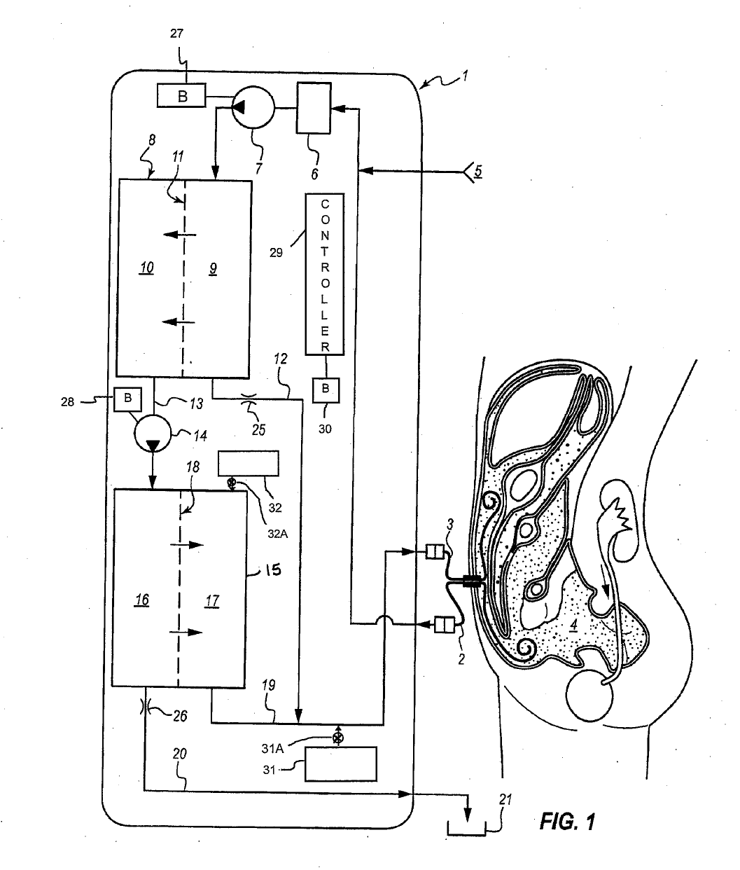

FIG. 1 is a schematic representation of the structure and function of one embodiment of the PD fluid reconstitution apparatus. On the right side of FIG. 1 is a representation of the body of a patient and the peritoneal space 4 is shown into which uptake 2 and return 3 segments of a PD catheter have been placed. In some implementations, all of the components of the system, with the exception of the PD catheter, are contained within an apparatus 1 (e.g. a sealed apparatus 1) located outside of the patient. Thus, apparatus 1 can have a housing that houses the components of the system, with the exception of the PD catheter. The distal segments of the uptake and return lumens of the PD catheter are ideally positioned at locations within the peritoneal space that are distant from each other. In this example, the uptake lumen is a curl shape and is located in the cul-de-sac of the pelvis and the distal segment of the return lumen is straight and located in Morrison's pouch under the free margin of the liver. Other arrangements are also contemplated.

Dialysate fluid from the peritoneal space is transported through an uptake lumen of the PD catheter by the action of a pump 7. The fluid initially passes through a preliminary filter 6, which removes particulate material, such as precipitated fibrin. In some implementations, it may be desirable for the filter 6 to have an average pore size to achieve a molecular weight cutoff (MWCO) of from about 100 to about 150 kDa. Filters of a variety of materials with such a MWCO are widely available (e.g., Millipore). In certain embodiments, the initial filter 6 or “prefilter” is designed to be easily replaceable once the function has been degraded by retained debris. The initial filter 6 can be arranged to filter out precipitated fibrin or mucoid materials from the dialysate fluid being removed from the peritoneal space, which materials may clog or otherwise degrade the performance of subsequent filters in the system.

In these or other embodiments herein, the pump (e.g. pump 7) can be any suitable pump, including for example an electrically powered pump such as peristaltic pump, a diaphragm pump, or a piston pump. In certain embodiments, the pump is powered by a brushless electric motor. In these or other motor driven pumps used herein, the it is preferred that the motor has the capacity to operate on a current draw of 2 amps or less while providing the pressures and flow rates desired for the PD process, including for example those preferred pressures and flow rates specified herein. The pump also desirably exhibits the capacity to operate on a voltage in the range of about 6 to about 24 volts. In some implementations, pump 7 or other pumps herein can be provided by a MG1000 Series Brushless Micropump, commercially available from TCS Micropumps Limited, United Kingdom, and in one specific illustration the pump can be provided by the MG1000F Brushless Micropump from TCS Micropumps.

In the illustrated embodiment, after passing through the pre-filtration provided by filter 6, the dialysate fluid passes into the high pressure side 9 of the first reverse osmotic or other filtration chamber 8. Here, the dialysate fluid comes into contact with a first reverse osmosis or other filtration membrane 11. This first membrane 11 contains pores which achieve a molecular weight cut off (MWCO), for example of approximately 15 kDa, sufficient to exclude the HMW component (e.g. Icodextrin) of the PD fluid. In the case of Icodextrin, the HMW component is a long chain starch molecule, for example with a range of molecular weights from 15 to 25 kDa. This first reverse osmosis membrane or other membrane may be made of one or more of a variety of commercially available materials, including, for example, cellulose, polysulfone, and polyethersulfone.

The action of the pump 7 generates sufficient pressure on the high pressure side 9 of the first chamber 8 so as to result in transit of some of the water and solute molecules which are below the MWCO across the membrane (forming a permeate) while the HMW osmotic component of the dialysate is constrained by the membrane to the high pressure side (in a retentate). The water and small molecules which do cross the first reverse osmosis membrane to the low pressure side 10 of the chamber 8 leave the first filtration chamber 8 through low pressure efferent lumen 13 in the permeate. Since this is not dead end filtration, most of the fluid, including most or all of the HMW osmotic component, leaves the high pressure segment of the first chamber through the high pressure efferent lumen 12 in the retentate. In order to maintain the necessary pressure in the first filtration chamber, an adjustable outflow restriction 25 is placed in the fluid path, in some embodiments. Later the contents of this high pressure efferent tube (retentate) will be combined with the free water product of the second filtration process and returned to the peritoneal space.

Persons of ordinary skill in the art will recognize that the use of “reverse osmotic filtration chamber” and “reverse osmosis membrane” in the passage above refer to the capacity of the filtration chamber 8 and its membrane 11 to substantially exclude the Icodextrin or other osmotic component of the dialysate (retaining it in the retentate) while driving water across the membrane 11 in opposition to the osmotic potential of the dialysate solution containing the osmotic component. Persons of ordinary skill will also recognize that this differs from and is more encompassing than some other usages relating to “reverse osmosis” membranes or processes which are well known to have and use pore sizes orders of magnitude smaller than those identified above so as to substantially exclude the passage of even small dissolved ions such as sodium while passing purified (e.g. desalinated) water.

The filter membrane 11 will typically have a pore size or molecular weight cutoff that is effective to generate a retentate that contains a predominant amount by weight (greater than 50% by weight) of the osmotic agent present in the used dialysate passed into the high pressure side 9 of the filter chamber 8. For these purposes the membrane will generally have a molecular weight cutoff that is lower than the weight average molecular weight of the osmotic agent, for example with the molecular weight cutoff for the filter 11 being no greater than 90% of the weight average molecular weight of the osmotic agent. In some embodiments, including but not limited to those in which the osmotic agent is Icodextrin, the filter membrane 11 can have a molecular weight cutoff in the range of about 3 kilodaltons (kDa) to about 15 kDa, more preferably in the range of about 5 kDa to about 12 kDa, and in a particular embodiment about 10 kDa. In addition or alternatively, the filter membrane 11 can have a surface area of at least about 20 cm², or at least about 50 cm², for example typically in the range of about 20 cm² to about 1000 cm² and more typically in the range of about 50 cm² to about 500 cm². In these or other embodiments identified herein, the filter membrane 11 is beneficially a polyethersulfone filter membrane. The first stage filter 11 can be provided, for example, by commercially available filter cartridges or other suitable filter devices. Illustratively, the first stage filter chamber 8 and its membrane 11 and other components can be provided by a crossflow ultrafiltration cassette, for example such as those available from Sartorius Stedim North America Inc. (Bohemia, N.Y., USA) under the tradename Vivaflow® (e.g. Vivaflow® 50, Vivaflow® 50R, or Vivaflow® 200). These and other filters and membranes enabling crossflow filtration, including crossflow ultrafiltration, to recover substantial amounts of the osmotic agent, can be used. These membranes can for example be hollow fiber membranes or flat sheet membranes (e.g. provided in filter chambers or cassettes as discussed above), with flat sheet membranes being preferred.

Icodextrin and other polymeric osmotic agents in fresh (unused) or in used condition can be a mixture of polymer molecules with varying molecular weights, which together establish the weight average molecular weight of the osmotic agent. Filtration by membrane 11 can result in selective passage (to the permeate) of lower molecular weight polymer molecules as compared to higher molecular weight polymer molecules of such an osmotic agent, and thus the weight average molecular weight of the retentate exiting the high pressure side 9 of the filter chamber 8 can be higher than that of the used dialysate passed into the high pressure side 9 of the filter chamber 8. The elimination of the lower molecular weight polymer molecules by their passage to the permeate, and the exclusion of those lower molecular weight polymer molecules from the regenerated dialysate fluid returned to the peritoneal cavity, may decrease the incidence of absorption of the Icodextrin or other osmotic agent by the patient from the peritoneal cavity, as smaller molecules are often absorbed more readily than larger molecules.

In some embodiments, the filter chamber 8 is operated at a pressure (at the high pressure side 9) in the range of about 15 pounds per square inch (psi) to about 100 psi, more preferably in the range of about 20 psi to about 50 psi, and most preferably in the range of about 20 psi to about 30 psi. In addition or alternatively, the total used dialysate throughput through the filter chamber 8 will be in the range of about 20 ml/minute to about 300 ml/minute, or about 50 ml/minute to about 200 ml/minute; and/or the ratio of the permeate flow in ml/minute to the retentate flow in ml/minute exiting the filter chamber 8 will be in the range of about 1:50 to about 1:10, or in the range of about 1:40 to about 1:15, or in the range of about 1:35 to about 1:20.

In certain embodiments, the retentate and the permeate resulting from the first filter chamber 8, and the effluents exiting the filter chamber 8 in effluent tubes 13 and 13, will have substantially equal (e.g. within 20% of one another, or within 10% of one another) concentrations of urea and creatinine (e.g. in mg/ml), with the first stage filter 8 thus not causing significant partitioning, or change in concentration, of these small molecules present in the spent dialysate removed from the peritoneal space of the patient. Nonetheless the creation of significant levels of permeate by first stage filter 11 will lead to the removal of significant amounts of urea, creatinine and potentially other wastes from the patient. In addition or alternatively, the retentate and the permeate resulting from the first stage filter chamber 8, and the effluents exiting filter chamber 8 in effluent tubes 12 and 13, can have substantially equal (e.g. within 20% of one another, or within 10% of one another) concentrations of sodium, magnesium, potassium, and/or calcium, and/or other electrolytes in the used dialysate withdrawn from the peritoneal space 4. While this may in some forms ultimately lead to some loss of these electrolyte(s), other components of the system can be provided to add amounts thereof to a regenerated dialysate to be returned to the peritoneal space 4 to partially or completely make up for the electrolyte(s) losses, and/or electrolytes can be administered (e.g. orally) to the patient to partially or completely make up for the electrolyte(s) losses. These and other variations will be apparent to those skilled in the field from the descriptions herein.

In preferred embodiments, the high pressure side 9 and the low pressure side 10 of filter chamber 8 are void space. Thus, all of the separation of components of the used dialysate caused by passage thereof into and out of the filter chamber 8 can be caused by the action of the membrane 11. This can facilitate beneficial flow of liquid through the filter chamber 8, and result in an unmodified retentate exiting filter chamber 8 through effluent tube 12 and an unmodified permeate exiting filter chamber through effluent tube 13.

However, in other embodiments, the high pressure side 9 and/or the low pressure side 10 can contain (e.g. be packed with) a particulate or other solid material that contacts and allows flow-through of liquid and that binds, selectively or non-selectively, one or more of anions, cations, waste, or other components of the liquid passing through the high pressure side 9 or low pressure side 10, respectively. Thus, this particulate or other solid material can modify the composition of the permeate or retentate generated by membrane 11 and thus provide a modified retentate and/or modified permeate that exits the filter chamber 8 through tube 12 and/or tube 13, respectively.

The water and small molecules which crossed the first membrane and exited the first chamber through the low pressure tube 13 are transported by a second pump 14 into the high pressure segment 16 of a second filtration chamber 15. In one alternative form, second pump 14 is omitted and its operations discussed below are instead effected by the fluid pressure generated by pump 7.

In the second reverse osmosis or other filtration chamber 15, water is separated from the nitrogen containing waste products of metabolism including urea, creatinine and uric acid as well as the group of waste products known as middle molecules by nanofiltration membrane 18. Membranes of this class include nonporous graphene and multilayer graphene oxide and rigid nanoporous silica membranes, as well as membranes comprised of tri-block polymers of polyisoprene-polystyrene-polydimethylacrylamide or of a polyamide film with an aramid support layer. In nanoporous reverse osmosis, separation is achieved primarily by molecular size. With sufficient pressure generated by pump 14 water crosses the membrane into the low pressure segment as a permeate while the larger waste products remain in the high pressure segment as a retentate. The fluid remaining in chamber 16 (the retentate) becomes a concentrated ultrafiltrate. The ultrafiltrate contains substantially all of the molecules present in the original peritoneal ultrafiltrate but is depleted of the HMW component and now is also significantly depleted of free water. The waste products leave through the high pressure efferent tube 20 in the retentate, and can flow to a discard container 21, for example a bag that can be worn by the patient. In order to maintain a high pressure an adjustable flow restriction 26 is placed on this outflow, in some embodiments. This outflow is in some embodiments collected in a drain bag and is discarded intermittently by the patient. In some modes of operation, in order to achieve 1-1.5 liter per 24 hours, an approximately six fold increase in concentration of the outflow in discard drain 20 is necessary compared to the concentration of the low pressure outflow 13 of the first reverse osmosis chamber.

Persons of ordinary skill in the art will recognize that the use of “reverse osmosis chamber” and “nonporous reverse osmosis” in the passages above relating to the second filtration chamber 15 refer to the capacity of the chamber 15 with its membrane 18 to substantially exclude the nitrogen containing waste products of metabolism including urea, creatinine and uric acid as well as the group of waste products known as middle molecules to concentrate them while driving water across the membrane in opposition to the osmotic potential of the solution (containing water and small molecules) which crossed the first membrane 11 and exited the first chamber 8 through the low pressure tube. Persons of ordinary skill will also recognize that this differs from and is more encompassing than some other usages relating to “reverse osmosis” membranes or processes as discussed above. The second filtration chamber 15 and its membrane preferably enable and are conducted to achieve crossflow nanofiltration of the liquid permeate from the first filtration chamber 8.

In certain embodiments, the membrane 18 will have a pore size in the range of about 2 to about 9 nanometers, and more typically about 3 to about 7 nanometers. In addition, the membrane 18 can exhibit the capacity to selectively retain urea molecules in the retentate while passing water molecules to the permeate. The filter 15 can be operated at any suitable pressure (at the input to the high pressure side 16) for these purposes and in some embodiments this pressure will be in the range of about 20 psi to about 100 psi.

The free water that crosses membrane 18 into the low pressure segment 17 of filter 15 exits through the low pressure efferent tube 19 as a permeate. This free water is combined with the contents of the high pressure efferent tube 12 (retentate) from the first chamber 8. This combined fluid is a reconstituted PD fluid that is then returned to the peritoneal space via the return limb 3 of the PD catheter. It will be understood by persons skilled in the field that membranes such as nanofiltration membranes discussed above for membrane 18 can also pass some amounts of small solutes, including but not limited to cations and/or anions, and that amounts of these small solutes can thus be contained in the water combined with the contents of high pressure efferent tube 12. In addition, while embodiments herein contemplate combining all of the water from the permeate of filter 15 with the retentate from filter 8, for example by combining the entire permeate from filter 15 with the retentate from filter 8, other modes of operation may be undertaken so that only a portion of the water from the permeate of filter 15 is so combined, for example where the permeate of filter 15 is further treated by filtration or otherwise to remove or separate components thereof.

In certain embodiments, also present is a recharging port for new PD fluid. The charging port can be located at any suitable position fluidly connecting to the fluid circuit in the PD system. One suitable location is shown as charging port 5 in FIG. 1. The HMW starch molecule does not remain permanently in the peritoneal space. Although the system is designed to reconstitute rather than discard the PD fluid, some loss of the starch molecules into the lymphatic system occurs in normal function of the peritoneal membrane. The half-life of the Icodextrin starch is between 12 and 18 hours. Therefore, in some implementations, 2 liters of Icodextrin are replenished on a daily basis.

The system 1 also preferably includes a battery 27 for electrically energizing pump 7 and a battery 28 for electrically energizing pump 14. Batteries 27 and 28 can be separate batteries, or can be provided by a single battery powering both pumps 7 and 14. The system 1 also in preferred embodiments includes a controller 29 for controlling the operation of system components including for example the pumps 7 and 14 and the valves or other similar devices providing restrictors 25 and/or 26, when present. Controller 29 can be provided by dedicated electrical circuitry and/or can be software-implemented using a microprocessor as controller 29. Controller 29 is electrically energized by a battery 30, which can be the same battery(ies) powering pumps 7 and 14 or can be a separate battery. In some embodiments, the battery or batteries powering pumps 7 and 14 and controller 30, and/or the controller 30, can be housed in the same system 1 housing along with pumps 7 and 14, filter chambers 8 and 15, and potentially also filter 6.

As discussed above, processing through filter or filtration chambers 8 and 15 may result in some loss of electrolytes or minerals such as calcium, magnesium, sodium and/or potassium, and/or buffering solutes such as lactate, acetate or bicarbonate, from the dialysate withdrawn from the peritoneal space 4. In one mode, to partially or completely make up for the loss(es), an aqueous electrolyte source 31 can be provided, and the aqueous electrolyte solution thereof can be metered or otherwise added into the regenerated dialysate in tube 19 for return to the peritoneal space, controlled for example by valve 31A positioned between source 31 and tube 19 that can be selectively opened or closed, and/or potentially also adjusted to various flow restriction levels. Valve 31A can in some forms be controlled by controller 29. Thus, this electrolyte source can include one, some or all of calcium, magnesium, sodium and potassium, and potentially also other electrolytes, minerals, nutrients, and/or possibly also therapeutic agents. In addition to or as an alternative to aqueous electrolyte source 31, system 1 can include an aqueous electrolyte source 32 that feeds into the low pressure (permeate) side 17 of the second filter chamber 15, to partially or completely make up for the loss(es) of electrolytes, minerals, buffers or other desired components in the stream 20 to be discarded. A valve 32A can be provided between electrolyte source 32 and the feed input into low pressure side 17 of chamber 15, to control the addition of electrolyte solution from source 32. As with valve 31A, valve 32A can be selectively opened or closed, and/or potentially also adjusted to various flow restriction levels, and can in some forms be controlled by controller 29. In some embodiments, aqueous electrolyte solution from source 32 can be metered or otherwise added to the permeate or low pressure side 17 of chamber 15, for example to flow co-current our counter-current to the retentate flow on retentate or high pressure side 16 of chamber 15, and can have a solute concentration sufficiently high to result in a forward osmotic gradient across membrane 18 from the retentate to the permeate side (i.e. so that the osmolality of the liquid on the permeate side of membrane 18 is higher than that of the liquid on the retentate side of the membrane 18). This can cause an osmotically driven passage of water from the retentate to the permeate side of membrane 18, resulting in an increased recovery of water in the permeate relative to that which would be caused by the pressure generated by pump 14 or any other pump pressurizing the liquid entering the high pressure side 16 of chamber 18. It will be understood in this regard that the input stream of electrolyte solution from source 32 for these purposes can generally be more concentrated in the electrolytes and/or other solute(s) than is desired for return to the peritoneal space 4, but that the added amounts of this electrolyte solution will be diluted by water passing through membrane 18 from chamber 16 to chamber 17 caused by the pressure exerted by pump 14 in combination with the forward osmotic pressure generated across membrane 18. In these modes of operation, advantageously, relatively low volumes of electrolyte solution from source 32 can be added (due to its concentrated nature). This can aide, for example, in minimizing the weight that must be supported by the patient when the source 32 is to be carried by the patient (e.g. as connected to or contained within the system 1 housing). Beneficially also, the use of forward osmotic pressure in chamber 15 can result in a greater amount of water passing through membrane 18 than would result from the pressure of pump 14 in the absence of the forward osmotic pressure, thus recovering more water for return to the peritoneal space in the regenerated dialysate and resulting in a more concentrated stream of wastes in line 20 to be discarded. It will be appreciated that in preferred embodiments, the source 31 and/or the source 32 will be configured to meter their respective solutions into the system, for example powered by a pump or pumps which in turn can be powered by a respective battery or batteries. The pump or pumps (and respective battery(ies) can be the same as that/those or different from those powering fluid flow or electrically energizing other components of the system 1.

Systems 1 are desirably relatively lightweight and wearable or otherwise portable by the patient. In certain embodiments, the weight of the system 1 housing and the components within the system 1 housing, will be less than 5 kg, more preferably less than 3 kg, and even more preferably less than 2 kg. For wearable systems 1, the housing and its components can be supported on the patient by a belt, harness, backpack, or any other suitable attachment member that can be worn around or over a body portion of the patient. As well, other wearable systems with these or other attachment members may have one or more than one housings or other support structures (typically rigid metal and/or plastic structures), that house or support different ones of the components of systems 1

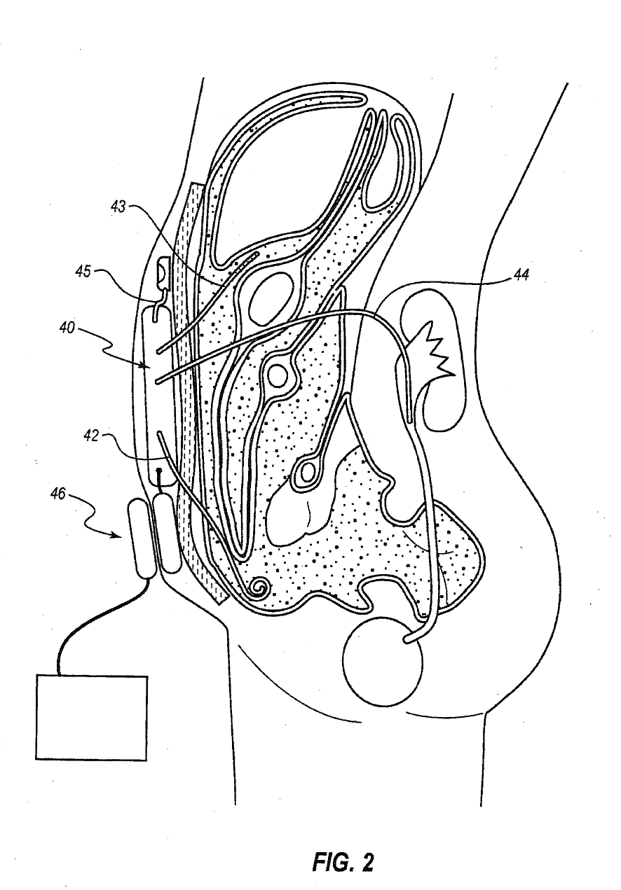

In FIG. 2, an implantable embodiment is depicted. The first and second reverse osmosis filtration chambers and the first and second pumps, as described with respect to the first illustrated embodiment of FIG. 1, are miniaturized and incorporated into a sealed and implantable device 40, which is shown implanted into the subcutaneous space of the abdominal wall. The uptake 42 and return 43 lumens of the PD catheter are shown attached to the implant and are positioned in the peritoneal space. The discard tube 44 is the high pressure efferent tube of the second reverse osmosis filtration chamber. As in the embodiment of FIG. 1, this discard tube 44 contains the concentrated waste products after the second reverse osmosis filtration step. However, in this embodiment, the tube 44 is implanted into one of the patient's ureters allowing the outflow to be drained continuously into the native renal collecting system and eliminated with normal urination. This catheter could also be implanted directly into the urinary bladder.

In the illustrated embodiment, the implantable device of FIG. 2 also contains a small internal battery. In various embodiments, recharging of the internal battery can be accomplished with inductive coupling 46, or through a small transcutaneous power cord.

Also shown in FIG. 2 is subcutaneous port 45 for adding additional PD fluid at regular intervals. In this embodiment this port is accessed through a subcutaneous needle puncture. These ports are widely used for venous vascular access, and thus the methods of implanting and using the ports is well known. However, the present disclosure is directed to the use of such ports for recharging an implanted PD system with PD fluid. As well, when electrolyte source 31 and/or electrolyte source 32 as in FIG. 1 are to be used in the system, these can for example be sources such as bags or other containers external of the patient and containing the electrolyte solution, and appropriate catheters, tubes or other ports can be percutaneously implanted in the patient to provide flow paths to their respective input locations in the implanted components of the system.

Systems such as that depicted in FIG. 2 can, in some instances, eliminate all catheters traversing the skin. No catheter tract is present to serve as a source of infection. The patient would be able to bathe, swim and shower. Additionally the lack of a catheter allows for greater work and other activities of daily living.

### LISTING OF CERTAIN EMBODIMENTS

The following is a non-limiting listing of embodiments disclosed herein:

Embodiment 1. A peritoneal dialysis method, comprising:
(i) removing a peritoneal dialysis ultrafiltrate from a peritoneal space of a patient, the peritoneal dialysis ultrafiltrate containing an osmotic agent, water, and nitrogen containing waste products of metabolism of the patient;
(ii) filtering particles from the peritoneal dialysis ultrafiltrate to form a pre-filtered peritoneal dialysis ultrafiltrate;
(iii) passing the pre-filtered peritoneal dialysis ultrafiltrate through a first filter to form a first retentate containing an amount of the osmotic agent and a first permeate containing water and nitrogen containing waste products of the patient;
(iv) passing the first permeate through a second filter to form a second retentate containing nitrogen containing waste products of the patient and a second permeate containing water;
(vi) combining at least a portion of the water contained in the second permeate with the first retentate to form a regenerated peritoneal dialysis medium containing an amount of the osmotic agent; and
(vii) returning the regenerated peritoneal dialysis medium to the peritoneal space of the patient.
   Embodiment 2. The peritoneal dialysis method of embodiment 1, wherein:
   during each of said filtering particles, said passing the pre-filtered peritoneal dialysis ultrafiltrate, said passing the first permeate, said combining and said returning, the first filter and the second filter are housed in a dialysis unit housing carried on the patient.
   Embodiment 3. The peritoneal dialysis method of embodiment 1 or 2, wherein:
said removing comprises first pumping the ultrafiltrate through a lumen of a catheter having a distal catheter region placed in the peritoneal space of the patient;
said filtering particles comprises second pumping the ultrafiltrate through a lumen having an in-line filter;
said first filter has a molecular weight cutoff in the range of about 5 to about 15 kDa; and
said returning comprises third pumping the regenerated peritoneal dialysis medium through a lumen of a catheter having a distal region positioned in the peritoneal space of the patient.
   Embodiment 4. The peritoneal dialysis method of embodiment 3, wherein:
said dialysis unit housing also houses a battery and one or more electric pumps electrically connected to and energizable by the battery; and
the one or more electric pumps power the first, second, and third pumping.
   Embodiment 5. The peritoneal dialysis method of embodiment 4, wherein at least one of the one or more electric pumps is powered by a brushless electric motor.
   Embodiment 6. The peritoneal dialysis method of any one of embodiments 1 to 5, wherein:
the osmotic agent comprises Icodextrin.
   Embodiment 7. The peritoneal dialysis method of any one of embodiments 1 to 6, wherein:
the first filter has a surface area in the range of about 20 to about 1000 cm².
   Embodiment 8. The peritoneal dialysis method of any one of embodiments 1 to 7, wherein:
first filter has a surface area in the range of about 50 to about 500 cm².
   Embodiment 9. The peritoneal dialysis method of any one of embodiments 1 to 8,wherein:
the first filter has a membrane comprising a polyether sulfone polymer.
   Embodiment 10. The peritoneal dialysis method of any one of embodiments 1 to 9, wherein:
the second filter has a membrane with a pore size in the range of about 2 nm to about 9 nm.

Embodiment 11. The peritoneal dialysis method of any one of embodiments 1 to 10, wherein:

said passing the pre-filtered peritoneal dialysis ultrafiltrate through a first filter is conducted so as to effect reverse osmosis filtration; and
said passing the first permeate through a second filter is conducted so as to effect reverse osmosis filtration.
Embodiment 12. The peritoneal dialysis method of any one of embodiments 1 to 10, wherein:
said passing the pre-filtered peritoneal dialysis ultrafiltrate through a first filter is conducted so as to effect crossflow filtration; and
the method also includes feeding an electrolyte solution into a permeate side of the second filter so as to create a forward osmotic gradient from a retentate side of the second filter to the permeate side of the second filter, the forward osmotic gradient causing an osmotically driven passage of water from the retentate side of the second filter to the permeate side of the second filter.
Embodiment 13. A peritoneal dialysis system, comprising:
a catheter for removing a peritoneal dialysis ultrafiltrate from a peritoneal space of a patient containing an osmotic agent, water, and nitrogen containing waste products of metabolism of the patient;
a filter arranged to filter particles from the peritoneal dialysis ultrafiltrate to form a pre-filtered peritoneal dialysis ultrafiltrate;
a first filter arranged to filter the pre-filtered peritoneal dialysis ultrafiltrate to form a first retentate containing the osmotic agent and a first permeate containing water and nitrogen containing waste products of the patient;
a second filter arranged to filter the first permeate to form a second retentate containing nitrogen containing waste products of the patient and a second permeate containing water; and
a catheter for returning a regenerated peritoneal dialysis medium containing the first retentate and at least a portion of the water contained in the second permeate to the peritoneal space of the patient.
   Embodiment 14. The peritoneal dialysis system of embodiment 13, also comprising:
   a wearable dialysis system housing that houses at least the first filter and the second filter.
   Embodiment 15. The peritoneal dialysis system of embodiment 14, wherein:
said wearable dialysis system housing also houses at least one battery and at least one electric pump electrically connected to and energizable by the battery.
   Embodiment 16. The peritoneal dialysis system of embodiment 15, wherein the electric pump is powered by a brushless electric motor.

Embodiment 17. The peritoneal dialysis system of any one of embodiments 13 to 16, wherein:
the first filter has a surface area the range of about 20 to about 1000 cm².
   Embodiment 18. The peritoneal dialysis system of any one of embodiments 13 to 17, wherein:
the second filter has a pore size in the range of about 2 nm to about 9 nm.
   Embodiment 19. The peritoneal dialysis method of any one of embodiments 13 to 18, wherein:
the first filter has a membrane comprising a polyether sulfone polymer.
   Embodiment 20. The peritoneal dialysis method of any one of embodiments 13 to 19, wherein:
the second filter has a membrane exhibiting a capacity to selectively retain urea while passing water.
   Embodiment 21. A method for forming a regenerated peritoneal dialysis fluid, comprising:
filtering particles from a peritoneal dialysis ultrafiltrate of a patient, the peritoneal dialysis ultrafiltrate containing an osmotic agent, water, and nitrogen containing waste products of metabolism of the patient, so as to form a pre-filtered peritoneal dialysis ultrafiltrate;
passing the pre-filtered peritoneal dialysis ultrafiltrate through a first filter to form a first retentate containing an amount of the osmotic agent and a first permeate containing water and nitrogen containing waste products of the patient;
passing the first permeate through a second filter to form a second retentate containing nitrogen containing waste products of the patient and a second permeate containing water; and
combining at least a portion of the water contained in the second permeate with the first retentate to form a regenerated peritoneal dialysis medium containing an amount of the osmotic agent.
   Embodiment 22. The method of embodiment 21, wherein:
   during each of said filtering particles, said passing the pre-filtered peritoneal dialysis ultrafiltrate, said passing the first permeate, and said combining, the first filter and the second filter are housed in a dialysis system housing carried on the patient.
   Embodiment 23. The peritoneal dialysis method of embodiment 21 or 22, wherein:
said filtering particles comprises pumping the ultrafiltrate through a lumen having an in-line filter; and
said first filter has a molecular weight cutoff in the range of about 5 to about 15 kDa.
   Embodiment 24. The peritoneal dialysis method of embodiment 23, wherein:
said dialysis unit housing also houses at least one battery and one or more electric pumps electrically connected to and energizable by the battery.
   Embodiment 25. The peritoneal dialysis method of embodiment 24, wherein at least one of the one or more electric pumps is powered by a brushless electric motor.
   Embodiment 26. The peritoneal dialysis method of any one of embodiments 21 to 25, wherein:
the osmotic agent comprises Icodextrin.
   Embodiment 27. The peritoneal dialysis method of any one of embodiments 21 to 26, wherein:
the first filter has a surface area in the range of about 20 to about 1000 cm².
   Embodiment 28. The peritoneal dialysis method of any one of embodiments 21 to 27, wherein:
the first filter has a surface area in the range of about 50 to about 500 cm².
   Embodiment 29. The peritoneal dialysis method of any one of embodiments 21 to 28, wherein:
the first filter has a membrane comprising a polyether sulfone polymer.
   Embodiment 30. The peritoneal dialysis method of any one of embodiments 21 to 29, wherein:
the second filter has a membrane having a pore size of about 2 nm to about 9 nm.
   Embodiment 31. The peritoneal dialysis method of any one of embodiments 21 to 30, wherein:
   said passing the pre-filtered peritoneal dialysis ultrafiltrate through a first filter is conducted so as to effect reverse osmosis filtration; and
   said passing the first permeate through a second filter is conducted so as to effect reverse osmosis filtration.
   Embodiment 32. The peritoneal dialysis method of any one of embodiments 21 to 30, wherein:
   said passing the pre-filtered peritoneal dialysis ultrafiltrate through a first filter is conducted so as to effect crossflow filtration; and
   the method also includes feeding an electrolyte solution into a permeate side of the second filter so as to create a forward osmotic gradient from a retentate side of the second filter to the permeate side of the second filter, the forward osmotic gradient causing an osmotically driven passage of water from the retentate side of the second filter to the permeate side of the second filter.
   Embodiment 33. A method for recapturing and reconstituting a high molecular weight peritoneal dialysis fluid, comprising:
filtering a dialysate fluid that has been removed from a peritoneal space of a patient to remove particulate material from the dialysate fluid, the dialysate fluid containing a high molecular weight component;
   after said filtering, pumping the dialysate fluid into a high pressure segment of a first filtration chamber so that the dialysate fluid comes into contact with a first membrane having a molecular weight cutoff;
   generating sufficient pressure in the high pressure segment of the first filtration chamber to result in transit of some of the water and solute molecules of the dialysate fluid that are below the molecular weight cutoff across the first membrane while the high molecular weight component of the dialysate fluid is constrained by the first membrane to the high pressure segment of the first filtration chamber, and wherein the water and solute molecules that transit across the first membrane exit the filtration chamber through a low pressure efferent lumen, and wherein the high molecular component constrained to the high pressure segment of the first membrane exits the filtration chamber with a fluid through a high pressure efferent lumen;
   pumping the water and solute molecules that exit the filtration chamber through the low pressure efferent lumen into a high pressure segment of a second filtration chamber and separating water from nitrogen containing waste products of metabolism by a nanofiltration membrane, with the water crossing the nanofiltration membrane to a low pressure segment of the second filtration chamber and exiting the second filtration chamber through a low pressure efferent lumen, and the nitrogen containing waste products that remained in the high pressure segment of the second filtration chamber exiting the second filtration chamber through a high pressure efferent lumen; and
   combining the water that exited the second filtration chamber through a low pressure efferent lumen with the fluid that exited the first filtration chamber through a high pressure efferent lumen to form a reconstituted peritoneal dialysis fluid.
   Embodiment 34. The method of embodiment 33, wherein the high molecular weight osmotic component is a starch.
   Embodiment 35. The method of embodiment 34, wherein the high molecular weight osmotic component is Icodextrin.
   Embodiment 36. The method of any one of embodiments 33 to 35, also comprising:
prior to said filtering, transporting the dialysis fluid from the peritoneal space of the patient through an uptake lumen of a peritoneal dialysis catheter by the action of a pump.
   37. The method of any one of embodiments 33 to 36, also comprising:
after said combining, returning the reconstituted peritoneal dialysis fluid to the peritoneal space of the patient through a return lumen of a peritoneal dialysis catheter.
   Embodiment 38. The method of any one of embodiments 33 to 38, wherein the first membrane is a reverse osmosis membrane having a molecular weight cutoff of approximately 15 kDa.
   Embodiment 39. The method of any one of embodiments 33 to 38, wherein the second filtration chamber achieves nanoporous reverse osmosis filtration.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the terms “about” or “approximately.” For each such reference, it is to be understood that, in some embodiments, the value, feature, or characteristic may be specified without approximation. For example, where qualifiers such as “about,” “substantially,” and “generally” are used, these terms include within their scope the qualified words in the absence of their qualifiers.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment, nor does any particular embodiment necessarily require all features disclosed.

1. A peritoneal dialysis method, comprising:
(i) removing a peritoneal dialysis ultrafiltrate from a peritoneal space of a patient, the peritoneal dialysis ultrafiltrate containing an osmotic agent, water, and nitrogen containing waste products of metabolism of the patient;
(ii) filtering particles from the peritoneal dialysis ultrafiltrate to form a pre-filtered peritoneal dialysis ultrafiltrate;
(iii) passing the pre-filtered peritoneal dialysis ultrafiltrate through a first filter to form a first retentate containing an amount of the osmotic agent and a first permeate containing water and nitrogen containing waste products of the patient;
(iv) passing the first permeate through a second filter to form a second retentate containing nitrogen containing waste products of the patient and a second permeate containing water;
(vi) combining at least a portion of the water contained in the second permeate with the first retentate to form a regenerated peritoneal dialysis medium containing an amount of the osmotic agent; and
(vii) returning the regenerated peritoneal dialysis medium to the peritoneal space of the patient.

2. The peritoneal dialysis method of claim 1, wherein:
during each of said filtering particles, said passing the pre-filtered peritoneal dialysis ultrafiltrate, said passing the first permeate, said combining and said returning, the first filter and the second filter are housed in a dialysis unit housing carried on the patient.

3. The peritoneal dialysis method of claim 1, wherein:
said removing comprises first pumping the ultrafiltrate through a lumen of a catheter having a distal catheter region placed in the peritoneal space of the patient;
said filtering particles comprises second pumping the ultrafiltrate through a lumen having an in-line filter;
said first filter has a molecular weight cutoff in the range of about 5 to about 15 kDa; and
said returning comprises third pumping the regenerated peritoneal dialysis medium through a lumen of a catheter having a distal region positioned in the peritoneal space of the patient.

4. The peritoneal dialysis method of claim 3, wherein:
said dialysis unit housing also houses a battery and one or more electric pumps electrically connected to and energizable by the battery; and
the one or more electric pumps power the first, second, and third pumping.

5. The peritoneal dialysis method of claim 4, wherein at least one of the one or more electric pumps is powered by a brushless electric motor.

6. The peritoneal dialysis method of claim 1, wherein:
the osmotic agent comprises Icodextrin.

7. The peritoneal dialysis method of claim 1, wherein:
the first filter has a surface area in the range of about 20 to about 1000 cm².

8. The peritoneal dialysis method of claim 1, wherein:
the first filter has a surface area in the range of about 50 to about 500 cm².

9. The peritoneal dialysis method of claim 1, wherein:
the first filter has a membrane comprising a polyether sulfone polymer.

10. The peritoneal dialysis method of claim 1, wherein:
the second filter has a membrane with a pore size in the range of about 2 nm to about 9 nm.

11. The peritoneal dialysis method of claim 1, wherein:
said passing the pre-filtered peritoneal dialysis ultrafiltrate through a first filter is conducted so as to effect reverse osmosis filtration; and
said passing the first permeate through a second filter is conducted so as to effect reverse osmosis filtration.

12. The peritoneal dialysis method of claim 1, wherein:
said passing the pre-filtered peritoneal dialysis ultrafiltrate through a first filter is conducted so as to effect crossflow filtration; and
the method also includes feeding an electrolyte solution into a permeate side of the second filter so as to create a forward osmotic gradient from a retentate side of the second filter to the permeate side of the second filter, the forward osmotic gradient causing an osmotically driven passage of water from the retentate side of the second filter to the permeate side of the second filter.

13. A peritoneal dialysis system, comprising:
a catheter for removing a peritoneal dialysis ultrafiltrate from a peritoneal space of a patient containing an osmotic agent, water, and nitrogen containing waste products of metabolism of the patient;
a filter arranged to filter particles from the peritoneal dialysis ultrafiltrate to form a pre-filtered peritoneal dialysis ultrafiltrate;
a first filter arranged to filter the pre-filtered peritoneal dialysis ultrafiltrate to form a first retentate containing the osmotic agent and a first permeate containing water and nitrogen containing waste products of the patient;
a second filter arranged to filter the first permeate to form a second retentate containing nitrogen containing waste products of the patient and a second permeate containing water; and
a catheter for returning a regenerated peritoneal dialysis medium containing the first retentate and at least a portion of the water contained in the second permeate to the peritoneal space of the patient.

14. The peritoneal dialysis system of claim 13, also comprising:
a wearable dialysis system housing that houses at least the first filter and the second filter.

15. The peritoneal dialysis system of claim 14, wherein:
said wearable dialysis system housing also houses at least one battery and at least one electric pump electrically connected to and energizable by the battery.

16. The peritoneal dialysis system of claim 15, wherein the electric pump is powered by a brushless electric motor.

17. The peritoneal dialysis system of claim 13, wherein:
the first filter has a surface area the range of about 20 to about 1000 cm².

18. The peritoneal dialysis system of claim 13, wherein:
the second filter has a pore size in the range of about 2 nm to about 9 nm.

19. The peritoneal dialysis method of claim 13, wherein:
the first filter has a membrane comprising a polyether sulfone polymer.

20. The peritoneal dialysis method of claim 13, wherein:
the second filter has a membrane exhibiting a capacity to selectively retain urea while passing water.

21. A method for forming a regenerated peritoneal dialysis fluid, comprising:
filtering particles from a peritoneal dialysis ultrafiltrate of a patient, the peritoneal dialysis ultrafiltrate containing an osmotic agent, water, and nitrogen containing waste products of metabolism of the patient, so as to form a pre-filtered peritoneal dialysis ultrafiltrate;
passing the pre-filtered peritoneal dialysis ultrafiltrate through a first filter to form a first retentate containing an amount of the osmotic agent and a first permeate containing water and nitrogen containing waste products of the patient;
passing the first permeate through a second filter to form a second retentate containing nitrogen containing waste products of the patient and a second permeate containing water; and
combining at least a portion of the water contained in the second permeate with the first retentate to form a regenerated peritoneal dialysis medium containing an amount of the osmotic agent.

22. The method of claim 21, wherein:
during each of said filtering particles, said passing the pre-filtered peritoneal dialysis ultrafiltrate, said passing the first permeate, and said combining, the first filter and the second filter are housed in a dialysis system housing carried on the patient.

23. The peritoneal dialysis method of claim 21, wherein:
said filtering particles comprises pumping the ultrafiltrate through a lumen having an in-line filter; and
said first filter has a molecular weight cutoff in the range of about 5 to about 15 kDa.

24. The peritoneal dialysis method of claim 23, wherein:
said dialysis unit housing also houses at least one battery and one or more electric pumps electrically connected to and energizable by the battery.

25. The peritoneal dialysis method of claim 24, wherein at least one of the one or more electric pumps is powered by a brushless electric motor.

26. The peritoneal dialysis method of claim 21, wherein:
the osmotic agent comprises Icodextrin.

27. The peritoneal dialysis method of claim 21, wherein:
the first filter has a surface area in the range of about 20 to about 1000 cm².

28-31. (canceled)

32. The peritoneal dialysis method of claim 21, wherein:
said passing the pre-filtered peritoneal dialysis ultrafiltrate through a first filter is conducted so as to effect crossflow filtration; and
the method also includes feeding an electrolyte solution into a permeate side of the second filter so as to create a forward osmotic gradient from a retentate side of the second filter to the permeate side of the second filter, the forward osmotic gradient causing an osmotically driven passage of water from the retentate side of the second filter to the permeate side of the second filter.

33. A method for recapturing and reconstituting a high molecular weight peritoneal dialysis fluid, comprising:
filtering a dialysate fluid that has been removed from a peritoneal space of a patient to remove particulate material from the dialysate fluid, the dialysate fluid containing a high molecular weight component;
after said filtering, pumping the dialysate fluid into a high pressure segment of a first filtration chamber so that the dialysate fluid comes into contact with a first membrane having a molecular weight cutoff;
generating sufficient pressure in the high pressure segment of the first filtration chamber to result in transit of some of the water and solute molecules of the dialysate fluid that are below the molecular weight cutoff across the first membrane while the high molecular weight component of the dialysate fluid is constrained by the first membrane to the high pressure segment of the first filtration chamber, and wherein the water and solute molecules that transit across the first membrane exit the filtration chamber through a low pressure efferent lumen, and wherein the high molecular component constrained to the high pressure segment of the first membrane exits the filtration chamber with a fluid through a high pressure efferent lumen;
pumping the water and solute molecules that exit the filtration chamber through the low pressure efferent lumen into a high pressure segment of a second filtration chamber and separating water from nitrogen containing waste products of metabolism by a nanofiltration membrane, with the water crossing the nanofiltration membrane to a low pressure segment of the second filtration chamber and exiting the second filtration chamber through a low pressure efferent lumen, and the nitrogen containing waste products that remained in the high pressure segment of the second filtration chamber exiting the second filtration chamber through a high pressure efferent lumen; and
combining the water that exited the second filtration chamber through a low pressure efferent lumen with the fluid that exited the first filtration chamber through a high pressure efferent lumen to form a reconstituted peritoneal dialysis fluid.

34. The method of claim 33, wherein the high molecular weight osmotic component is a starch.

35. The method of claim 34, wherein the high molecular weight osmotic component is Icodextrin.

36. The method of claim 33, also comprising:
prior to said filtering, transporting the dialysis fluid from the peritoneal space of the patient through an uptake lumen of a peritoneal dialysis catheter by the action of a pump.

37. The method of claim 33, also comprising:
after said combining, returning the reconstituted peritoneal dialysis fluid to the peritoneal space of the patient through a return lumen of a peritoneal dialysis catheter.

38. The method of claim 33, wherein the first membrane is a reverse osmosis membrane having a molecular weight cutoff of approximately 15 kDa.

39. The method of claim 33, wherein the second filtration chamber achieves nanoporous reverse osmosis filtration.
